(19) United States
(12) Patent Application Publication
(10) Pub. No.: US 2024/0108466 A1
(43) Pub. Date: Apr. 4, 2024

(54) PROSTHETIC HEART VALVE DEVICES AND ASSOCIATED SYSTEMS AND METHODS

(71) Applicant: Twelve, Inc., Redwood City, CA (US)

(72) Inventors: Richard Francis, Pine Springs, MN (US); Scott Robertson, Portland, OR (US); Marian Lally, Galway (IE); Alexander Hill, Blaine, MN (US); Katherine Miyashiro, Portland, OR (US); Paraic Frisby, Galway (IE); James R. Keogh, Maplewood, MN (US)

(21) Appl. No.: 18/541,105

(22) Filed: Dec. 15, 2023

Related U.S. Application Data
(62) Division of application No. 16/944,957, filed on Jul. 31, 2020, now Pat. No. 11,877,926.
Application No. 16/944,957 is a division of application No. 15/642,834, filed on Jul. 6, 2017, now Pat. No. 10,729,541.

(51) Int. Cl. A61F 2/24

(52) U.S. Cl. CPC A61F 2/2418; A61F 2/2412; A61F 2210/0014; A61F 2250/0067; A61F 2250/0068; A61F 2250/0098

(57) ABSTRACT

The present technology is a prosthetic heart valve device, and related systems and methods, for treating a native valve of a human heart having a native annulus and native leaflets. One embodiment comprises a valve support, a prosthetic valve assembly within the valve support, and an anchoring member having an upstream portion and a downstream portion. The device further includes an extension member coupled to the fixation frame and extending radially outward therefrom. The extension member includes a plurality of discrete fabric segments having a first end at the anchoring member and a second end spaced apart from the anchoring member, wherein each discrete fabric segment includes lateral edges extending from the first end to the second end of the discrete fabric segment. The discrete fabric segments are configured to move circumferentially relative to each other.

This application is a division of U.S. patent application Ser. No. 16/944,957, filed Jul. 31, 2020, which is a division of U.S. patent application Ser. No. 15/642,834, filed Jul. 6, 2017, now U.S. Pat. No. 10,729,541, the entirety of both of which are hereby incorporated by reference.

### CROSS-REFERENCE TO RELATED APPLICATION(S)

The present application incorporates the subject matter of (1) International Patent Application No. PCT/US2014/029549, filed Mar. 14, 2014, (2) International Patent Application No. PCT/US2012/061219, filed Oct. 19, 2012, (3) International Patent Application No. PCT/US2012/061215, filed Oct. 19, 2012, (4) International Patent Application No. PCT/US2012/043636, filed Jun. 21, 2012. The present application also incorporates the subject matter of U.S. application Ser. No. 15/490,047, filed Apr. 18, 2017, and U.S. application Ser. No. 15/643,011 (Attorney Docket No. C00013493.USU2), also filed concurrently herewith.

### TECHNICAL FIELD

The present technology relates generally to prosthetic heart valve devices. In particular, several embodiments are directed to prosthetic mitral valves and devices for percutaneous repair and/or replacement of native mitral valves and associated systems and methods.

### BACKGROUND

Heart valves can be affected by several conditions. For example, mitral valves can be affected by mitral valve regurgitation, mitral valve prolapse and mitral valve stenosis. Mitral valve regurgitation is abnormal leaking of blood from the left ventricle into the left atrium caused by a disorder of the heart in which the leaflets of the mitral valve fail to coapt into apposition at peak contraction pressures. The mitral valve leaflets may not coapt sufficiently because heart diseases often cause dilation of the heart muscle, which in turn enlarges the native mitral valve annulus to the extent that the leaflets do not coapt during systole. Abnormal backflow can also occur when the papillary muscles are functionally compromised due to ischemia or other conditions. More specifically, as the left ventricle contracts during systole, the affected papillary muscles do not contract sufficiently to effect proper closure of the leaflets.

Mitral valve prolapse is a condition when the mitral leaflets bulge abnormally up in to the left atrium. This can cause irregular behavior of the mitral valve and lead to mitral valve regurgitation. The leaflets may prolapse and fail to coapt because the tendons connecting the papillary muscles to the inferior side of the mitral valve leaflets (chordae tendineae) may tear or stretch. Mitral valve stenosis is a narrowing of the mitral valve orifice that impedes filling of the left ventricle in diastole.

Mitral valve regurgitation is often treated using diuretics and/or vasodilators to reduce the amount of blood flowing back into the left atrium. Surgical approaches (open and intravascular) for either the repair or replacement of the valve have also been used to treat mitral valve regurgitation. For example, typical repair techniques involve cinching or resecting portions of the dilated annulus. Cinching, for example, includes implanting annular or peri-annular rings that are generally secured to the annulus or surrounding tissue. Other repair procedures suture or clip the valve leaflets into partial apposition with one another.

Alternatively, more invasive procedures replace the entire valve itself by implanting mechanical valves or biological tissue into the heart in place of the native mitral valve. These invasive procedures conventionally require large open thoracotomies and are thus very painful, have significant morbidity, and require long recovery periods. Moreover, with many repair and replacement procedures, the durability of the devices or improper sizing of annuloplasty rings or replacement valves may cause additional problems for the patient. Repair procedures also require a highly skilled cardiac surgeon because poorly or inaccurately placed sutures may affect the success of procedures.

Less invasive approaches to aortic valve replacement have been implemented in recent years. Examples of pre-assembled, percutaneous prosthetic valves include, e.g., the CoreValve Revalving® System from Medtronic/Corevalve Inc. (Irvine, CA, USA) and the Edwards-Sapien® Valve from Edwards Lifesciences (Irvine, CA, USA). Both valve systems include an expandable frame and a tri-leaflet bioprosthetic valve attached to the expandable frame. The aortic valve is substantially symmetric, circular, and has a muscular annulus. The expandable frames in aortic applications have a symmetric, circular shape at the aortic valve annulus to match the native anatomy, but also because tri-leaflet prosthetic valves require circular symmetry for proper coaptation of the prosthetic leaflets. Thus, aortic valve anatomy lends itself to an expandable frame housing a replacement valve since the aortic valve anatomy is substantially uniform, symmetric, and fairly muscular. Other heart valve anatomies, however, are not uniform, symmetric or sufficiently muscular, and thus transvascular aortic valve replacement devises may not be well suited for other types of heart valves.

The tricuspid valve on the right side of the heart, although it normally has three leaflets, poses similar challenges to less invasive treatment as the mitral valve. Therefore there is a need for a better prosthesis to treat tricuspid valve disease as well.

Given the difficulties associated with current procedures, there remains the need for simple, effective, and less invasive devices and methods for treating dysfunctional heart valves.

### BRIEF DESCRIPTION OF THE DRAWINGS

Many aspects of the present disclosure can be better understood with reference to the following drawings. The components in the drawings are not necessarily to scale, and instead emphasis is placed on illustrating clearly the principles of the present disclosure. Furthermore, components can be shown as transparent in certain views for clarity of illustration only and not to indicate that the illustrated component is necessarily transparent. For ease of reference, throughout this disclosure identical reference numbers and/or letters are used to identify similar or analogous components or features, but the use of the same reference number does not imply that the parts should be construed to be identical. Indeed, in many examples described herein, identically numbered components refer to different embodiments that are distinct in structure and/or function. The headings provided herein are for convenience only.

FIG. 1 is a schematic, cross-sectional illustration of the heart showing an antegrade approach to the native mitral valve from the venous vasculature in accordance with various embodiments of the present technology.

FIG. 2 is a schematic, cross-sectional illustration of the heart showing access through the inter-atrial septum (IAS) maintained by the placement of a guide catheter over a guidewire in accordance with various embodiments of the present technology.

FIGS. 3 and 4 are schematic, cross-sectional illustrations of the heart showing retrograde approaches to the native mitral valve through the aortic valve and arterial vasculature in accordance with various embodiments of the present technology.

FIG. 5 is a schematic, cross-sectional illustration of the heart showing an approach to the native mitral valve using a trans-apical puncture in accordance with various embodiments of the present technology.

FIG. 6A is a cross-sectional side view and FIG. 6B is a top view schematically illustrating a prosthetic heart valve device in accordance with an embodiment of the present technology.

FIG. 7A is a top isometric view of a prosthetic heart valve device in accordance with an embodiment of the present technology.

FIG. 7B is an enlarged view of a strand of the support member of the prosthetic heart valve device shown in FIG. 7A.

FIG. 7C is a cross-sectional view of the strand shown in FIG. 7A taken along line 7C-7C.

FIG. 8A is a schematic top view of a prosthetic heart valve device in a deployed configuration configured in accordance with an embodiment of the present technology.

FIG. 8B is a schematic end view of the prosthetic heart valve device of FIG. 8A in a delivery configuration positioned within a delivery catheter configured in accordance with an embodiment of the present technology.

FIGS. 8C-8E are cross-sectional side views of a portion of the prosthetic heart valve device shown in FIGS. 8A and 8B during deployment in accordance with an embodiment of the present technology.

FIGS. 9A and 9B are end views of three isolated segments of the extension member of the prosthetic heart valve device of FIG. 8A, shown in overlapping and extended configurations, respectively.

FIGS. 9C and 9D are end views of three isolated segments of an extension member of a prosthetic heart valve device, shown in overlapping and extended configurations, respectively.

FIGS. 9E and 9F are end views of three isolated segments of an extension member of a prosthetic heart valve device, shown in overlapping and extended configurations, respectively.

FIG. 10 is a schematic isometric view of a prosthetic heart valve device in accordance with another embodiment of the present technology.

### DETAILED DESCRIPTION

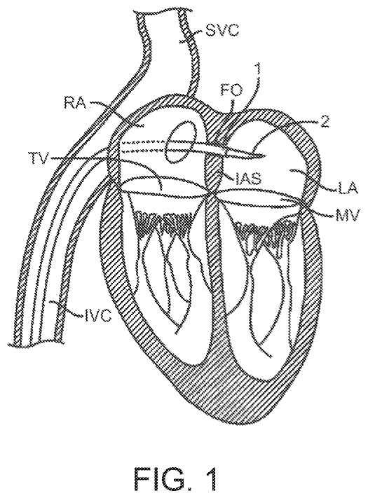

Specific details of several embodiments of the technology are described below with reference to FIGS. 1-10. Although many of the embodiments are described below with respect to prosthetic valve devices, systems, and methods for percutaneous replacement of a native mitral valve, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-10.

With regard to the terms “distal” and “proximal” within this description, unless otherwise specified, the terms can reference a relative position of the portions of a prosthetic valve device and/or an associated delivery device with reference to an operator and/or a location in the vasculature or heart. For example, in referring to a delivery catheter suitable to deliver and position various prosthetic valve devices described herein, “proximal” can refer to a position closer to the operator of the device or an incision into the vasculature, and “distal” can refer to a position that is more distant from the operator of the device or further from the incision along the vasculature (e.g., the end of the catheter). With respect to a prosthetic heart valve device, the terms “proximal” and “distal” can refer to the location of portions of the device with respect to the direction of blood flow. For example, proximal can refer to an upstream position or a location where blood flows into the device (e.g., inflow region), and distal can refer to a downstream position or a location where blood flows out of the device (e.g., outflow region).

### Overview

Several embodiments of the present technology are directed to mitral valve replacement devices that address the unique challenges of percutaneously replacing native mitral valves and are well-suited to deliver a therapeutic agent to the anatomy. Compared to replacing aortic valves, percutaneous mitral valve replacement faces unique anatomical obstacles that render percutaneous mitral valve replacement significantly more challenging than aortic valve replacement. First, unlike relatively symmetric and uniform aortic valves, the mitral valve annulus has a non-circular D-shape or kidney-like shape, with a non-planar, saddle-like geometry often lacking symmetry. The complex and highly variable anatomy of mitral valves makes it difficult to design a mitral valve prosthesis that conforms well to the native mitral annulus of specific patients. As a result, the prosthesis may not fit well with the native leaflets and/or annulus, which can leave gaps that allows backflow of blood to occur. For example, placement of a cylindrical valve prosthesis in a native mitral valve may leave gaps in commissural regions of the native valve through which perivalvular leaks may occur.

Current prosthetic valves developed for percutaneous aortic valve replacement are unsuitable for use in mitral valves. First, many of these devices require a direct, structural connection between the stent-like structure that contacts the annulus and/or leaflets and the prosthetic valve. In several devices, the stent posts which support the prosthetic valve also contact the annulus or other surrounding tissue. These types of devices directly transfer the forces exerted by the tissue and blood as the heart contracts to the valve support and the prosthetic leaflets, which in turn distorts the valve support from its desired cylindrical shape. This is a concern because most cardiac replacement devices use tri-leaflet valves, which require a substantially symmetric, cylindrical support around the prosthetic valve for proper opening and closing of the three leaflets over years of life. As a result, when these devices are subject to movement and forces from the annulus and other surrounding tissues, the prostheses may be compressed and/or distorted causing the prosthetic leaflets to malfunction. Moreover, a diseased mitral annulus is much larger than any available prosthetic aortic valve. As the size of the valve increases, the forces on the valve leaflets increase dramatically, so simply increasing the size of an aortic prosthesis to the size of a dilated mitral valve annulus would require dramatically thicker, taller leaflets, and might not be feasible.

In addition to its irregular, complex shape, which changes size over the course of each heartbeat, the mitral valve annulus lacks a significant amount of radial support from surrounding tissue. Compared to aortic valves, which are completely surrounded by fibro-elastic tissue that provides sufficient support for anchoring a prosthetic valve, mitral valves are bound by muscular tissue on the outer wall only. The inner wall of the mitral valve anatomy is bound by a thin vessel wall separating the mitral valve annulus from the inferior portion of the aortic outflow tract. As a result, significant radial forces on the mitral annulus, such as those imparted by an expanding stent prostheses, could lead to collapse of the inferior portion of the aortic tract. Moreover, larger prostheses exert more force and expand to larger dimensions, which exacerbates this problem for mitral valve replacement applications.

The chordae tendineae of the left ventricle may also present an obstacle in deploying a mitral valve prosthesis. Unlike aortic valves, mitral valves have a maze of cordage under the leaflets in the left ventricle that restrict the movement and position of a deployment catheter and the replacement device during implantation. As a result, deploying, positioning and anchoring a valve replacement device on the ventricular side of the native mitral valve annulus is complicated.

Embodiments of the present technology provide systems, methods and apparatus to treat heart valves of the body, such as the mitral valve, that address the challenges associated with the anatomy of the mitral valve and provide for delivery of a therapeutic agent to the local anatomy. The apparatus and methods enable a percutaneous approach using a catheter delivered intravascularly through a vein or artery into the heart, or through a cannula inserted through the heart wall. For example, the apparatus and methods are particularly well-suited for trans-septal approaches, but can also be trans-apical, trans-atrial, and direct aortic delivery of a prosthetic replacement valve to a target location in the heart. Additionally, the embodiments of the devices and methods as described herein can be combined with many known surgeries and procedures, such as known methods of accessing the valves of the heart (e.g., the mitral valve or triscuspid valve) with antegrade or retrograde approaches, and combinations thereof.

### Access to the Mitral Valve

To better understand the structure and operation of valve replacement devices in accordance with the present technology, it is helpful to first understand approaches for implanting the devices. The mitral valve or other type of atrioventricular valve can be accessed through the patient's vasculature in a percutaneous manner. By percutaneous it is meant that a location of the vasculature remote from the heart is accessed through the skin, typically using a surgical cut down procedure or a minimally invasive procedure, such as using needle access through, for example, the Seldinger technique. The ability to percutaneously access the remote vasculature is well known and described in the patent and medical literature. Depending on the point of vascular access, access to the mitral valve may be antegrade and may rely on entry into the left atrium by crossing the inter-atrial septum (e.g., a trans-septal approach). Alternatively, access to the mitral valve can be retrograde where the left ventricle is entered through the aortic valve. Access to the mitral valve may also be achieved using a cannula via a trans-apical approach. Depending on the approach, the interventional tools and supporting catheter(s) may be advanced to the heart intravascularly and positioned adjacent the target cardiac valve in a variety of manners, as described herein.

FIG. 1 illustrates a stage of a trans-septal approach for implanting a valve replacement device. In a trans-septal approach, access is via the inferior vena cava IVC or superior vena cava SVC, through the right atrium RA, across the inter-atrial septum IAS, and into the left atrium LA above the mitral valve MV. As shown in FIG. 1, a catheter 1 having a needle 2 moves from the inferior vena cava IVC into the right atrium RA. Once the catheter 1 reaches the anterior side of the inter-atrial septum IAS, the needle 2 advances so that it penetrates through the septum, for example at the fossa ovalis FO or the foramen ovale into the left atrium LA. At this point, a guidewire replaces the needle 2 and the catheter 1 is withdrawn.

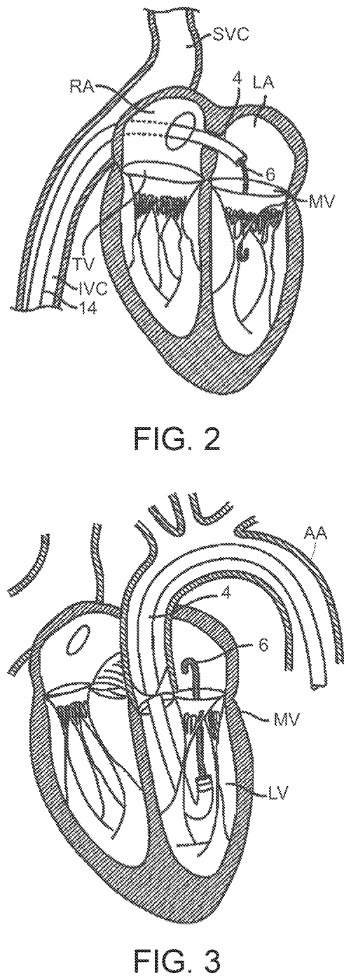

FIG. 2 illustrates a subsequent stage of a trans-septal approach in which guidewire 6 and guide catheter 4 pass through the inter-atrial septum IAS. The guide catheter 4 provides access to the mitral valve for implanting a valve replacement device in accordance with the technology.

In an alternative antegrade approach (not shown), surgical access may be obtained through an intercostal incision, preferably without removing ribs, and a small puncture or incision may be made in the left atrial wall. A guide catheter passes through this puncture or incision directly into the left atrium, sealed by a purse string-suture.

The antegrade or trans-septal approach to the mitral valve, as described above, can be advantageous in many respects. For example, antegrade approaches will usually enable more precise and effective centering and stabilization of the guide catheter and/or prosthetic valve device. The antegrade approach may also reduce the risk of damaging the chordae tendinae or other subvalvular structures with a catheter or other interventional tool. Additionally, the antegrade approach may decrease risks associated with crossing the aortic valve as in retrograde approaches. This can be particularly relevant to patients with prosthetic aortic valves, which cannot be crossed at all or without substantial risk of damage.

FIGS. 3 and 4 show examples of a retrograde approaches to access the mitral valve. Access to the mitral valve MV may be achieved from the aortic arch AA, across the aortic valve AV, and into the left ventricle LV below the mitral valve MV. The aortic arch AA may be accessed through a conventional femoral artery access route or through more direct approaches via the brachial artery, axillary artery, radial artery, or carotid artery. Such access may be achieved with the use of a guidewire 6. Once in place, a guide catheter 4 may be tracked over the guidewire 6. Alternatively, a surgical approach may be taken through an incision in the chest, preferably intercostally without removing ribs, and placing a guide catheter through a puncture in the aorta itself. The guide catheter 4 affords subsequent access to permit placement of the prosthetic valve device, as described in more detail herein. Retrograde approaches advantageously do not need a trans-septal puncture. Cardiologists also more commonly use retrograde approaches, and thus retrograde approaches are more familiar.

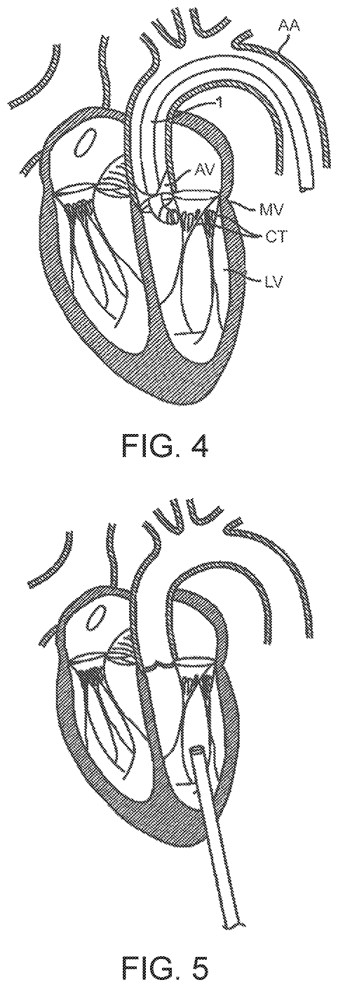

FIG. 5 shows a trans-apical approach via a trans-apical puncture. In this approach, access to the heart is via a thoracic incision, which can be a conventional open thoracotomy or sternotomy, or a smaller intercostal or sub-xyphoid incision or puncture. An access cannula is then placed through a puncture in the wall of the left ventricle at or near the apex of the heart. The catheters and prosthetic devices of the invention may then be introduced into the left ventricle through this access cannula. The trans-apical approach provides a shorter, straighter, and more direct path to the mitral or aortic valve. Further, because it does not involve intravascular access, the trans-apical approach does not require training in interventional cardiology to perform the catheterizations required in other percutaneous approaches.

### Selected Embodiments of Prosthetic Heart Valve Devices and Methods

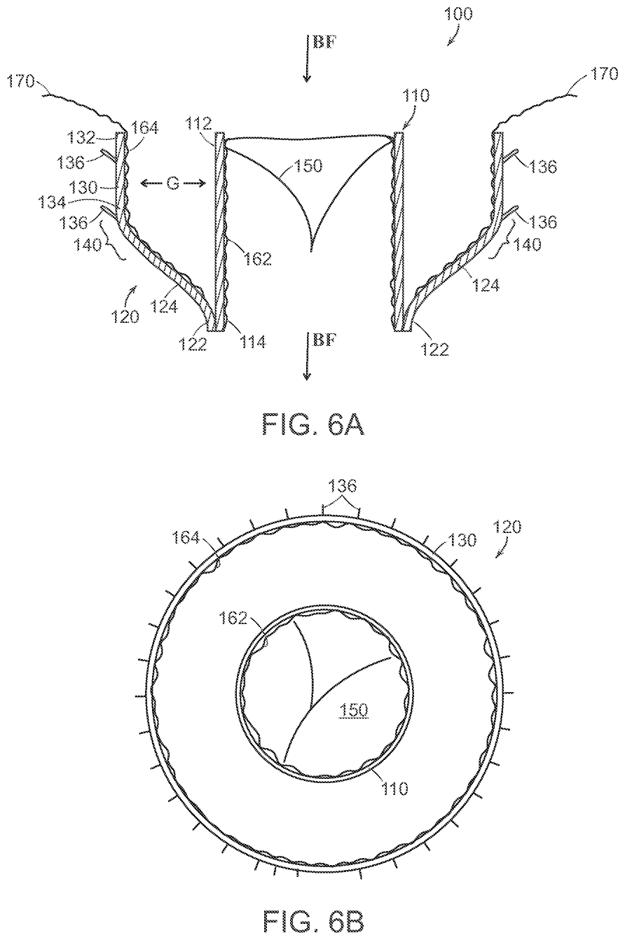

Embodiments of the present technology can treat one or more of the valves of the heart, and in particular several embodiments advantageously treat the mitral valve. The prosthetic valve devices of the present technology can also be suitable for replacement of other valves (e.g., a bicuspid or tricuspid valve) in the heart of the patient. Examples of prosthetic heart valve devices in accordance with embodiments of the present technology are described in this section with reference to FIGS. 6A-10. Specific elements, substructures, advantages, uses, and/or other features of the embodiments described with reference to FIGS. 6A-10 can be suitably interchanged, substituted or otherwise configured with one another. Furthermore, suitable elements of the embodiments described with reference to FIGS. 6A-10 can be used as stand-alone and/or self-contained devices.

FIG. 6A is a side cross-sectional view and FIG. 6B is a top plan view of a prosthetic heart valve device (“device”) 100 in accordance with an embodiment of the present technology. The device 100 includes a valve support 110, an anchoring member 120 attached to the valve support 110, and a prosthetic valve assembly 150 within the valve support 110. Referring to FIG. 6A, the valve support 110 has an inflow region 112 and an outflow region 114. The prosthetic valve assembly 150 is arranged within the valve support 110 to allow blood to flow from the inflow region 112 through the outflow region 114 (arrows BF), but prevent blood from flowing in a direction from the outflow region 114 through the inflow region 112.

In the embodiment shown in FIG. 6A, the anchoring member 120 includes a base 122 attached to the outflow region 114 of the valve support 110 and a plurality of arms 124 projecting laterally outward from the base 122. The anchoring member 120 also includes a fixation structure 130 extending from the arms 124. The fixation structure 130 can include a first portion 132 and a second portion 134. The first portion 132 of the fixation structure 130, for example, can be an upstream region of the fixation structure 130 that, in a deployed configuration as shown in FIG. 6A, is spaced laterally outward apart from the inflow region 112 of the valve support 110 by a gap G. The second portion 134 of the fixation structure 130 can be a downstream-most portion of the fixation structure 130. The fixation structure 130 can be a cylindrical ring (e.g., straight cylinder or conical), and the outer surface of the fixation structure 130 can define an annular engagement surface configured to press outwardly against the native annulus. The fixation structure 130 can further include a plurality of fixation elements 136 that project radially outward and are inclined toward an upstream direction. The fixation elements 136, for example, can be barbs, hooks, or other elements that are inclined only in the upstream direction (e.g., a direction extending away from the downstream portion of the device 100).

Referring still to FIG. 6A, the anchoring member 120 has a smooth bend 140 between the arms 124 and the fixation structure 130. For example, the second portion 134 of the fixation structure 130 extends from the arms 124 at the smooth bend 140. The arms 124 and the fixation structure 130 can be formed integrally from a continuous strut or support element such that the smooth bend 140 is a bent portion of the continuous strut. In other embodiments, the smooth bend 140 can be a separate component with respect to either the arms 124 or the fixation structure 130. For example, the smooth bend 140 can be attached to the arms 124 and/or the fixation structure 130 using a weld, adhesive or other technique that forms a smooth connection. The smooth bend 140 is configured such that the device 100 can be recaptured in a capsule or other container after the device 100 has been at least partially deployed.

The device 100 can further include a first sealing member 162 on the valve support 110 and a second sealing member 164 on the anchoring member 120. The first and second sealing members 162, 164 can be made from a flexible material, such as Dacron® or another type of polymeric material. The first sealing member 162 can cover the interior and/or exterior surfaces of the valve support 110. In the embodiment illustrated in FIG. 6A, the first sealing member 162 is attached to the interior surface of the valve support 110, and the prosthetic valve assembly 150 is attached to the first sealing member 162 and commissure portions of the valve support 110. The second sealing member 164 is attached to the inner surface of the anchoring member 120. As a result, the outer annular engagement surface of the fixation structure 130 is not covered by the second sealing member 164 so that the outer annular engagement surface of the fixation structure 130 directly contacts the tissue of the native annulus.

The device 100 can further include an extension member 170. The extension member 170 can be an extension of the second sealing member 164, or it can be a separate component attached to the second sealing member 164 and/or the first portion 132 of the fixation structure 130. The extension member 170 can be a flexible member that, in a deployed state as shown in FIG. 6A, flexes relative to the first portion 132 of the fixation structure 130. In operation, the extension member 170 guides the device 100 during implantation such that the device is located at a desired elevation and centered relative to the native annulus. As described below, the extension member 170 can include a support member, such as a metal wire or other structure, that can be visualized during implantation and/or is configured to deliver a therapeutic agent.

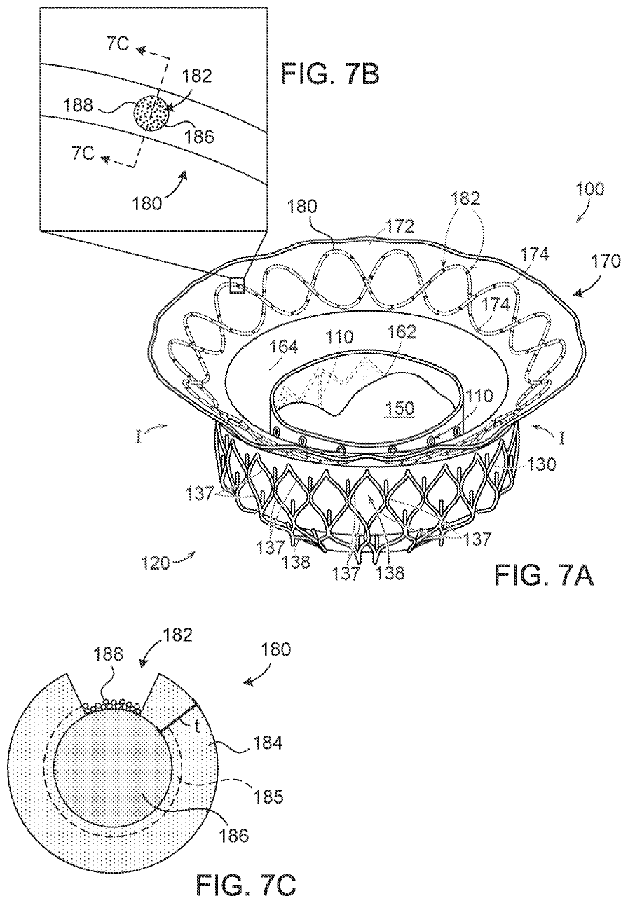

FIG. 7A is a top isometric view of an example of the device 100. In this embodiment, the valve support 110 defines a first frame (e.g., an inner frame) and fixation structure 130 of the anchoring member 120 defines a second frame (e.g., an outer frame) that each include a plurality of structural elements. The fixation structure 130, more specifically, includes structural elements 137 arranged in diamond-shaped cells 138 that together form at least a substantially cylindrical ring when freely and fully expanded as shown in FIG. 7A. The structural elements 137 can be struts or other structural features formed from metal, polymers, or other suitable materials that can self-expand or be expanded by a balloon or other type of mechanical expander.

Several embodiments of the fixation structure 130 can be a generally cylindrical fixation ring having an outwardly facing engagement surface. For example, in the embodiment shown in FIG. 7A, the outer surfaces of the structural elements 137 define an annular engagement surface configured to press outwardly against the native annulus in the deployed state. In a fully expanded state without any restrictions, the fixation structure 130 is at least substantially parallel to the valve support 110. However, the fixation structure 130 can flex inwardly (arrow I) in the deployed state when it presses radially outwardly against the inner surface of the native annulus of a heart valve.

The embodiment of the device 100 shown in FIG. 7A includes the first sealing member 162 lining the interior surface of the valve support 110, and the second sealing member 164 along the inner surface of the fixation structure 130. The extension member 170 has a flexible web 172 (e.g., a fabric) and a support member 174 comprising a plurality of strands 180 (e.g., a wire, a strut, a cable, a ribbon, a fiber, etc.) attached to the flexible web 172. The flexible web 172 can extend from the second sealing member 164 without a metal-to-metal connection between the fixation structure 130 and the support member 174. For example, the extension member 170 can be a continuation of the material of the second sealing member 164. Several embodiments of the extension member 170 are thus a floppy structure that can readily flex with respect to the fixation structure 130.

FIG. 7B is an enlarged view of a portion of one of the strands 180 of the support member 174 shown in FIG. 7A, and FIG. 7C is a cross-sectional view of the strand 180 shown in FIG. 7B taken along line 7C-7C. Referring to FIGS. 7B and 7C together, at least a portion of the strands 180 can include an inner core 186 surrounded by an outer material 184 having a thickness t. The outer material 184 can be a first material, and the inner core 186 can be a second material different than the first material. For example, in some embodiments at least a portion of the strands 180 are drawn-filled tubes (“DFT's”) formed of at least two dissimilar materials such that the strand 180 includes the desired physical and mechanical attributes of each of the dissimilar materials. In the embodiment shown in FIGS. 7A-7C, the outer material 184 is a superelastic alloy (e.g., Nitinol, cobalt-chromium, MP35N®, etc.) that provides structural support to the strand 180, and the inner core 186 is a radiopaque material (e.g., platinum, tantalum, tungsten, palladium, gold, silver, etc.) that improves visualization of the support member 174 and/or provides additional structural support to the strand 180.

In certain embodiments, the strands 180 further include an optional intermediate material 185 (FIG. 7C, shown in dashed lines) positioned between the outer material 184 and the inner core 186. In such embodiments, the inner core material and the outer material 184 may be the same, and the intermediate material 185 may be a different material. For example, the outer material 184 and the inner core material may be a first material, and the intermediate material 185 may be a second material different than the first material. For instance, the outer material 184 and the inner core material 186 may be a superelastic alloy (e.g., Nitinol, cobalt-chromium, etc.), and the intermediate material 185 may be a radiopaque material (e.g., platinum, tantalum, palladium, gold, etc.). In other embodiments, the outer material 184, the inner core material, and the intermediate material 185 are different materials. In yet other embodiments, the strand 180 may include more than three layers of material.

As shown in FIGS. 7B and 7C, at least a portion of the strands 180 include a plurality of recesses 182 extending through at least a portion of the thickness t of the outer material 184, and one or more therapeutic agents 188 in the recesses 182. In some embodiments, one or more of the recesses 182 may have a depth that extends through the entire thickness t of the outer material 184 such that a top surface of the inner core 186 is exposed through the recess 182. In certain embodiments, one or more of the recesses 182 may extend through the entire thickness of the outer material 184 and a portion of the thickness of the inner core 186 such that the recess 182 terminates at a depth within the inner core 186. In either of the foregoing embodiments, the therapeutic agent 188 may be positioned on the inner core 186, or the inner core 186 may be impregnated with the therapeutic agent 188. In other embodiments, the recess 182 extends through only a portion of the thickness t of the outer material 184 such that the inner core 186 is not exposed and the recess 182 terminates at an intermediate depth within the outer material 184. In such embodiments, the therapeutic agent 188 may be positioned on the exposed inner surface of the outer material 184. Additionally, the strand 180 may include recesses that extend to different depths within the outer material 184 and/or the inner core 186. Moreover, in those embodiments including an intermediate material 185, the strand 180 may include one or more first recesses that extend through the outer layer and expose the intermediate material 185, and one or more second recesses that extend through the outer material 184 and the intermediate material 185 and expose the inner core 186. In some embodiments, the recesses 182 are through-holes that pass through the complete cross-sectional dimension (e.g., diameter) of the strands 180. In any of the foregoing embodiments, the therapeutic agent 188 may occupy all or a portion of the recess 182.

Although the recesses 182 shown in FIGS. 7A-7C have a circular cross-sectional shape, in other embodiments one or more of the recesses may have other suitable cross-sectional shapes (e.g., a square, an oval, a rectangle, etc.). Likewise, one or more of the recesses 182 can have the same cross-sectional area or different cross-sectional areas at different depths of the recess. For example, one or more of the recesses 182 may have a cross-sectional area that tapers towards a bottom surface of the recess. Also, at least some of the recesses 182 may be positioned at the same circumferential position along the strand and/or at least some of the recesses 182 may have different circumferential positions. Moreover, in some embodiments multiple recesses may be positioned at the same axial location along the strand 180. In other embodiments, all of the recesses 182 are positioned at different axial locations along the strand.

Suitable therapeutic agents 188 may include one or more drugs and/or bioactive agents that reduce inflammation at the annulus and promote ingrowth between the extension member 170 and one or more of the leaflets and/or the atrial floor. As such, incorporation of one or more therapeutic agents 188 can increase tolerance and incorporation of the prosthetic heart valve device and/or reduce para-valvular leakage. Examples of suitable anti-inflammatory drugs include paclitaxel, sirolimus, and the like. Examples of bioactive agents include one or more growth factors, such as platelet-derived growth factor (“PDGF”), fibroblast growth factor (“FGF”), transforming growth factor (“TGF”), and other suitable mitogens that stimulate growth of endothelial cells, smooth muscle cells, and fibroblasts.

In some embodiments, the therapeutic agent 188 may be configured to have a controlled delivery rate over a desired period of time. For example, the therapeutic agent 188 may be associated with (e.g., encapsulated by, bound to, etc.) a delivery vehicle, such as a bioresorbable-polymer blend, for timed-release of the therapeutic agent 188. In certain embodiments, the web may be impregnated with a bioactive agent or a bioactive agent and delivery vehicle to control the rate of release of the bioactive agent.

While the embodiment illustrated in FIG. 7A illustrates a generally concentric arrangement of the strands 180 forming a crisscrossing serpentine pattern (e.g., zig-zag pattern, diamond pattern), other arrangements and patterns of the one or more strands 180 (e.g., serpentine, wavy, radial arms, square, etc.) can also be formed to provide a desired rigidity to the extension member 170. Moreover, although the support member 174 shown in FIG. 7A is separate from the fixation structure 130, in other embodiments the support member 174 is integral with the fixation structure 130. In addition, the support member 174, the valve support 110, and/or the anchoring member 120 may include, as described above, a mixture of materials, alloys, struts, wires, strand types and/or strands sizes. For example, in some embodiments a portion of the structure, struts or strands may be DFT's, and another portion of the structure, struts, or strands may be made of a polymer fiber and/or a metal wire. In particular embodiments, the extension member comprises an annular fabric web and a braided superelastic material (e.g., Nitinol) in the hem of the fabric or as strips perpendicular to the circumference of the annular web. In any of the embodiments disclosed herein, the extension member includes a platinum-impregnated fabric and/or may include a thin-film superelastic material (e.g., Nitinol) deposited onto the fabric. In addition, any of the structural members disclosed herein may include one or more laser cut Nitinol strips (e.g., straight strips, curved strips, etc.). In some embodiments, one or more portions of the structural member, the support member 174, the valve support 110, and/or the anchoring member 120 may have one or more holes riveted with radiopaque markers.

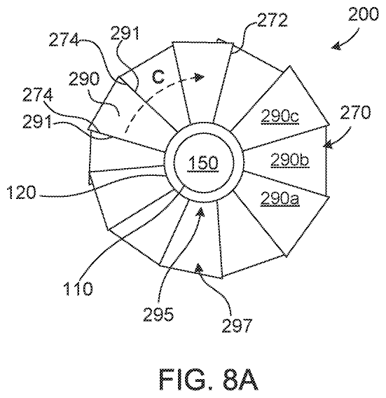

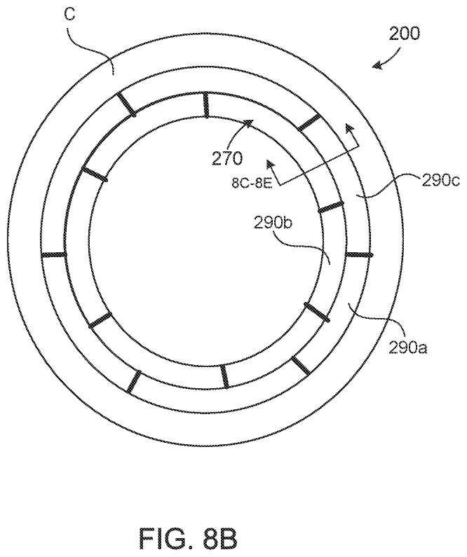

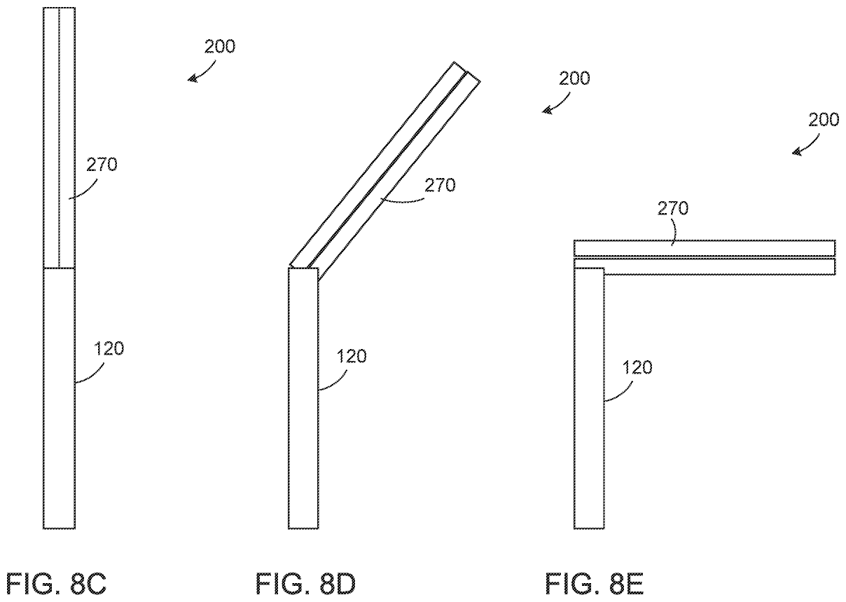

FIG. 8A is a schematic top view of another embodiment of a prosthetic heart valve device 200 (“the device 200”) in a deployed configuration, and FIG. 8B is a schematic end view of the device 200 in a delivery configuration positioned within a delivery catheter C lumen. FIGS. 8C-8E are cross-sectional views of the device 200 taken along line 8C-8E (FIG. 8B) as the device 200 is deploying (delivery catheter C not shown in FIGS. 8C-8E for ease of illustration). The device 200 may include several components that are generally similar in structure and function to those of the device 100 in FIGS. 6A-7C. For example, the device 200 can include the valve support 110 (FIG. 8A), the anchoring member 120 (FIG. 8A), the prosthetic valve assembly 150 (FIG. 8A), and the second sealing member 164 (not visible in FIGS. 8A-8E), all of which are generally similar to those discussed above with reference to FIGS. 6A-7C. As such, common acts and structures and/or sub-structures are identified by the same reference numbers, and only significant differences in operation and structure are described below.

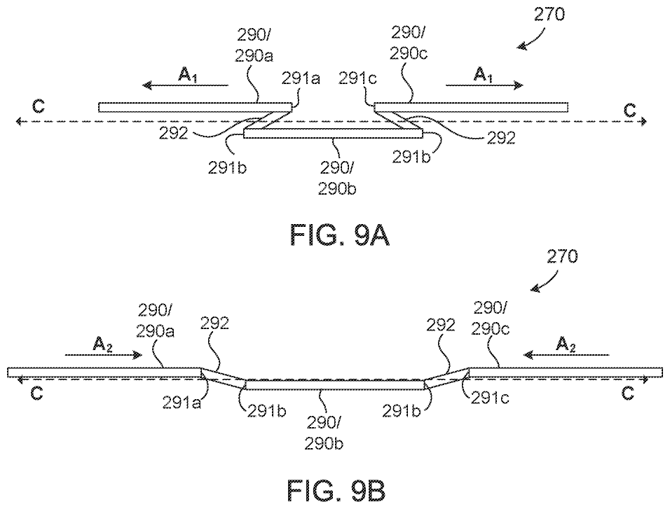

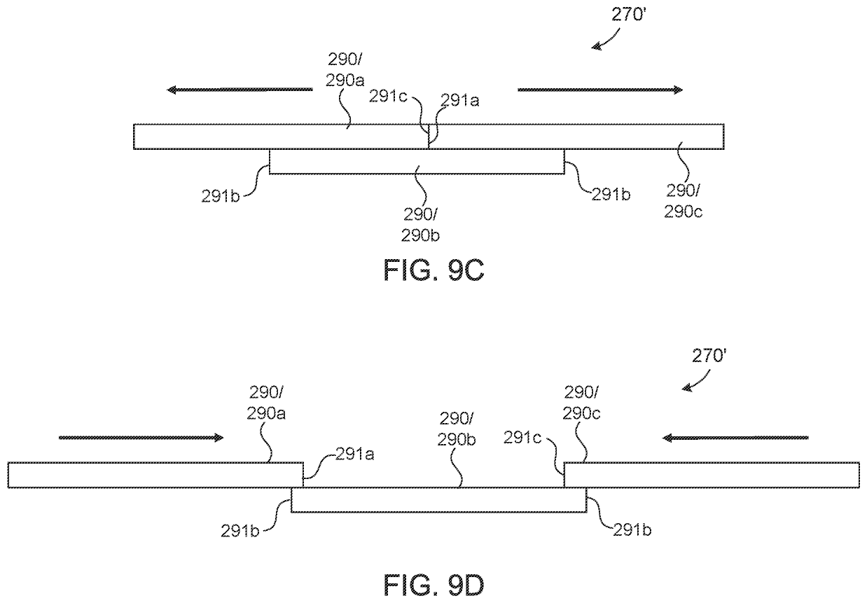

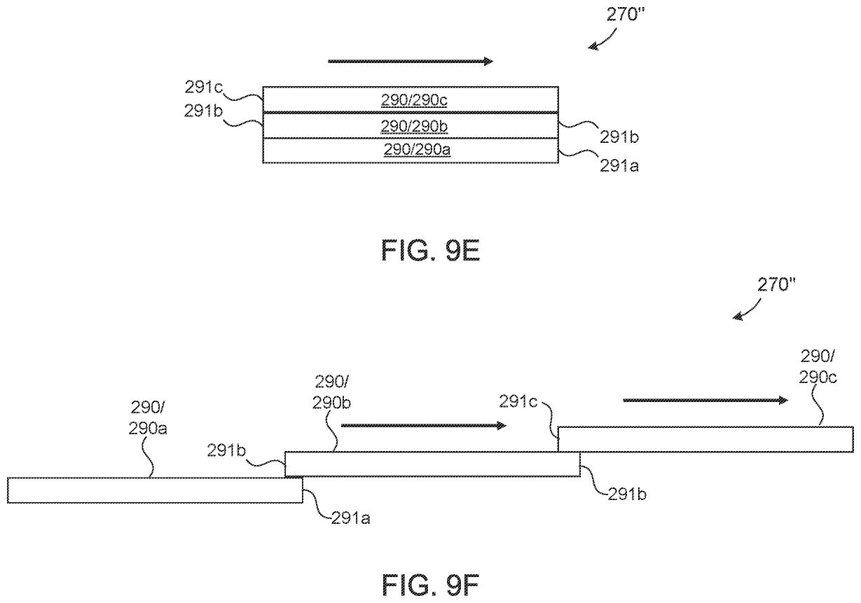

Referring to FIG. 8A, the device 200 includes an extension member 270 having a plurality of discrete fabric segments 290 around its circumference (only one segment 290 is labeled in FIG. 8 for ease of illustration), each having a first end portion 295 at the anchoring member 120, a second end portion 297 spaced apart from the anchoring member 120, and a width that extends between opposing edges 291. In some embodiments, adjacent segments 290 may be coupled along their respective edges 291 by a flexible portion 292 (see FIGS. 9A and 9B) such that the edges 291 are free to slide with respect to one another.

FIGS. 9A and 9B are end views of three adjacent, isolated segments (referred to as “first-third segments 290a-c,” respectively) shown in overlapping and extended configurations, respectively. In an overlapping configuration (FIG. 9A), edge 291a of the first segment 290a and edge 291c of the third segment 290c overlap the adjacent edges 291b of the second segment 290b. In response to one or more forces exerted on the extension member 270 by the surrounding anatomy, at least a portion of the first-third segments 290a-c may move in circumferentially opposite directions away from one another (indicated by arrows A1 in FIG. 9A) such that edges 291a and 291c slide relative to edges 291b in a circumferential direction C. Depending on the exerted force, edges 291a and 291c may slide past the corresponding adjacent edge 291b such that the first-third segments 290a-c are in an extended configuration (FIG. 9B) in which edges 291a and 291c are spaced apart from the corresponding adjacent edge 291b along the circumference C of the extension member 270. In some embodiments, each of the edges 291a and 291c may be spaced apart from the corresponding adjacent edge 291b by no more than the length of the flexible portion 292. When the first-third segments 290a-c are in the extended configuration and one or more forces are exerted on the extension member 270 by the surrounding anatomy, at least a portion of the first-third segments 290a-c may move in circumferentially opposite directions towards one another (indicated by arrows A2 in FIG. 9B).

In some embodiments, the extension member 270 and/or segments 290 can have other configurations. For example, FIGS. 9C and 9D are end views of three isolated segments 290a-290c (collectively “the segments 290”) of an extension member 270′ configured in accordance with another embodiment of the present technology, shown in overlapping and extended configurations, respectively. Likewise, FIGS. 9E and 9F are end views of three isolated segments 290a-290c (collectively “the segments 290”) of another extension member 270″ configured in accordance with the present technology, shown in overlapping and extended configurations, respectively. In some embodiments, such as those shown in FIGS. 9C-9F, the segments 290 are not coupled along their respective edges 291 by a flexible portion 292. When the device 200 is positioned within the annulus, the plurality of segments 290 may move with respect to one another to adapt to the natural geometry of the annulus and/or adjust their relative positions as necessary while the extension member 270 seals with the local cardiac anatomy.

In some embodiments the individual segments 290 include support members 274 (e.g., metal or polymer strands) extending along and/or defining at least a portion of their individual edges, and a flexible web 272 (e.g., a fabric) may extend between the support members 274 of each segment 290. In such embodiments, the flexible portions 292 may comprise a separate flexible web extending between edges 291 of adjacent segments 290. In other embodiments, the extension member 270 and/or individual segments 290 may have other suitable configurations. For example, in some embodiments the individual segments 290 include support members 274 (e.g., metal or polymer strands) extending radially outward from the fixation structure 130 wherein each individual segment 290 has at least one support member covered by a flexible web 272 (e.g., a fabric) extending from each support member 274. In some embodiments, at least a portion of the flexible web 272 of each individual segment 290 overlaps at least a portion of the flexible web 272 of an adjacent individual segment member 290. In some embodiments, the flexible web 272 extends between the support members 274 of each segment 290. In some embodiments, extension member 270 comprises a flexible web 272 that is continuous around the fixation structure 130. For example, in one embodiment, the flexible web 272 may comprise one or more pockets wherein individual support members 290 are free to move or slide within the one or more pockets. In one embodiment, the number of flexible web pockets equals the number of individual support members. In an alternative embodiment, the number of pockets is less than the number of individual support members. For example, in one embodiment, all of the individual support members reside within a single pocket.

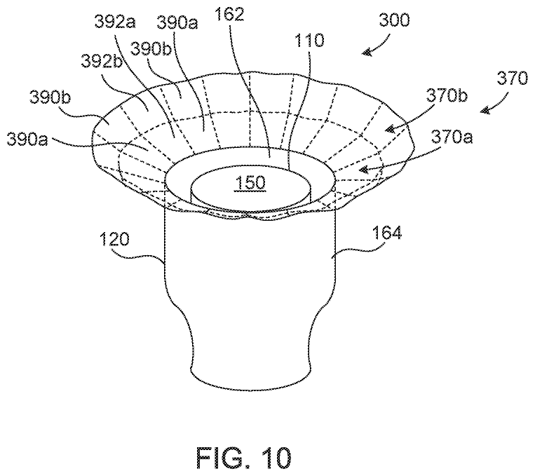

FIG. 10 is a schematic isometric view of a prosthetic heart valve device 300 in accordance with another embodiment of the present technology. The device 300 may include several components that are generally similar in structure and function to those of the device 100 in FIGS. 6A-7C. For example, the device 300 can include the valve support 110, the anchoring member 120, the prosthetic valve assembly 150, and the second sealing member 164, all of which are generally similar to those discussed above with reference to FIGS. 6A-7C. As such, common acts and structures and/or sub-structures are identified by the same reference numbers, and only significant differences in operation and structure are described below.

In the embodiment shown in FIG. 10, the extension member 370 includes a first annular portion 370a extending radially outwardly from the anchoring member 120, and a second annular portion 370b extending radially outwardly from the first annular portion 370a. The first annular portion 370a may have a plurality of discrete segments 390a and flexible portions 392 (only one of each labeled) similar to the segments 290 and flexible portions 292 of the extension member 270 shown in FIGS. 8-9B. The second annular portion 370b may also have a plurality of discrete segments 390b and flexible portions 392b (only one of each labeled) similar to the segments 290 and flexible portions 292 of the extension member 270 shown in FIGS. 8-9B. As shown in FIG. 10, in some embodiments the segments 390b of the second annular portion 370b are circumferentially aligned with the segments 390b of the first annular portion 370a, and the flexible portions 392b of the second annular portion 370b are circumferentially aligned with the flexible portions 392a of the first annular portion 370a.

### EXAMPLES

The following examples are illustrative of several embodiments of the present technology:
1. A prosthetic heart valve device comprising:
an anchoring member having an annular fixation frame with an upstream portion and a downstream portion;
a tubular valve support having a first portion coupled to the downstream portion of the fixation frame and a second portion spaced radially inward from the upstream portion of the fixation frame;
a valve assembly coupled to the valve support and having at least one leaflet movable from a closed position in which blood flow is blocked through the valve support and an open position in which blood flow is allowed through the valve support in a downstream direction; and
an extension member coupled to the fixation frame and extending radially outward therefrom, wherein the extension member includes a plurality of wires, wherein at least a portion of the wires include an inner core surrounded by an outer material, and wherein (a) the inner core is a first material, (b) the outer material is a second material different than the first material and has a thickness, (c) the wires include a plurality of recesses extending through at least a portion of the thickness of the outer material, and (d) a therapeutic agent in the recesses for delivery to the anatomy when the prosthetic heart valve device is positioned at a native annulus.
2. The device of example 1 wherein the upstream portion of the fixation frame is radially deformable without substantially deforming the second portion of the tubular valve support.
3. The device of example 1 or example 2 wherein the extension member further includes a sealing member extending along at least a portion of its length, and wherein the wires are coupled to the sealing member.
4. The device of any one of examples 1-3 wherein the wires are integral with the upstream portion of the fixation frame.
5. The device of any one of examples 1-3 wherein the wires are mechanically isolated from the fixation frame.
6. The device of any one of examples 1-5 wherein the first material is radiopaque, and the second material is a superelastic alloy.
7. The device of any one of examples 1-6 wherein the therapeutic agent includes at least one of an anti-inflammatory drug and a bioactive agent.
8. The device of any one of examples 1-7 wherein at least some of the plurality of recesses extend through the entire thickness of the outer material, thereby exposing at least a portion of the inner core.
9. The device of any one of examples 1-8 wherein at least some of the plurality of recesses extend through the entire thickness of the outer material and at least a portion of the thickness of the inner core such that the at least some of the plurality of recesses terminate at an intermediate depth within the inner core.
10. The device of any one of examples 1-9 wherein the extension member further comprises an intermediate material positioned between the inner core and the outer material.
11. The device of example 10 wherein at least some of the recesses extend through the entire thickness of the outer material, thereby exposing at least a portion of the intermediate material.
12. The device of example 10 wherein at least some of the recesses extend through the entire thickness of the outer material and the entire thickness of the intermediate material, thereby exposing at least a portion of the inner core.
13. A prosthetic heart valve device comprising:
an anchoring member having an annular fixation frame with an upstream portion and a downstream portion;
a tubular valve support having a first portion coupled to the downstream portion of the anchoring member and a second portion spaced radially inward from the upstream portion of the anchoring member;
a valve assembly coupled to the valve support and having at least one leaflet movable from a closed position in which blood flow is blocked through the valve support and an open position in which blood flow is allowed through the valve support in a downstream direction; and
an extension member coupled to the fixation frame and extending radially outward therefrom, wherein the extension member includes a plurality of wires, wherein at least a portion of the wires include an inner core surrounded by an outer material, and wherein (a) the inner core is a first material, (b) the outer material is a second material different than the first material and has a thickness, (c) the wires include a plurality of recesses extending through at least a portion of the thickness of the outer material, and (d) a radiopaque material in the recesses.
14. The device of example 13 wherein the upstream portion of the fixation frame is radially deformable without substantially deforming the second portion of the tubular valve support.
15. The device of example 13 or example 14 wherein the extension member further includes a sealing member extending along at least a portion of its length, and wherein the wires are coupled to the sealing member.
16. The device of any one of examples 13-15 wherein the wires are integral with the upstream portion of the fixation frame.
17. The device of any one of examples 13-15 wherein the wires are mechanically isolated from the fixation frame.
18. The device of any one of examples 13-17 wherein the first material is radiopaque, and the second material is a superelastic alloy.
19. The device of any one of examples 13-18 wherein the therapeutic agent includes at least one of an anti-inflammatory drug and a bioactive agent.
20. The device of any one of examples 13-19 wherein at least some of the plurality of recesses extend through the entire thickness of the outer material, thereby exposing at least a portion of the inner core.
21. The device of any one of examples 13-20 wherein at least some of the plurality of recesses extend through the entire thickness of the outer material and at least a portion of the thickness of the inner core such that the at least some of the plurality of recesses terminate at an intermediate depth within the inner core.
22. The device of any one of examples 13-21 wherein the extension member further comprises an intermediate material positioned between the inner core and the outer material.
23. The device of example 22 wherein at least some of the recesses extend through the entire thickness of the outer material, thereby exposing at least a portion of the intermediate material.
24. The device of example 22 wherein at least some of the recesses extend through the entire thickness of the outer material and the entire thickness of the intermediate material, thereby exposing at least a portion of the inner core.
27. A prosthetic heart valve device comprising:
an anchoring member having a radially expandable frame with an interior and having an upstream portion and a downstream portion, wherein the upstream portion includes a tissue fixation portion configured to press outwardly against tissue located at and/or downstream of a native annulus of a heart valve in a subject and configured to be at least partially deformable to conform to a shape of the tissue;
a valve positioned relative to the anchoring member and having at least one leaflet movable from a closed position in which blood flow is blocked through the interior and an open position in which blood flow is allowed through the interior in a flow direction from the upstream portion toward the downstream portion, wherein the valve is spaced inwardly apart from the tissue fixation portion of the anchoring member such that the valve remains competent when the tissue fixation portion is deformed to conform to the shape of the tissue; and
an extension assembly extending laterally away from the upstream portion of the anchoring member in a deployed configuration, wherein the extension member comprises a plurality of discrete fabric segments having a first end at the anchoring member and a second end spaced apart from the anchoring member, wherein the fabric segments are arranged side-by-side around the extension member and adjacent fabric segments are loosely coupled together by a flexible coupling means such that the second ends of adjacent fabric segments can be configured independently of each other in the deployed state.
28. The prosthetic heart valve device of example 27 wherein the segments do not include a structural member.
29. The prosthetic heart valve device of example 27 or example 28 wherein the extension member is configured to deform relative to the expandable frame in the deployed configuration.
30. The prosthetic heart valve device of any one of examples 27-29 wherein the coupling means is elastic.
31. The prosthetic heart valve device of any one of examples 27-29 wherein the coupling means is a suture.
32. The prosthetic heart valve device of any one of examples 27-31 wherein the distal termini of the segments coincide with the second end of the extension member.
33. The prosthetic heart valve device of any one of examples 27-32 wherein the segments are spaced apart from the expandable frame such that the segments are only indirectly coupled to the anchoring member.
34. The prosthetic heart valve device of any one of examples 27-32 wherein the segments are directly connected to the expandable frame.

From the foregoing, it will be appreciated that specific embodiments of the invention have been described herein for purposes of illustration, but that various modifications may be made without deviating from the scope of the invention. For example, several individual components can be interchange with each other in the different embodiments. Accordingly, the invention is not limited except as by the appended claims.

I/We claim:

1. A prosthetic heart valve device comprising:
an anchoring member having an annular fixation frame, the fixation frame including a first fixation end and a second fixation end;
a tubular valve support having a first portion coupled to the second end of the fixation frame and a second portion spaced radially inward from the first end of the fixation frame;
a valve assembly coupled to the valve support and having at least one leaflet movable from a closed position in which blood flow is blocked through the valve support and an open position in which blood flow is allowed through the valve support in a downstream direction; and
an extension member coupled to the fixation frame and extending radially outward therefrom, wherein the extension member comprises a plurality of discrete fabric segments having a first end at the anchoring member and a second end spaced apart from the anchoring member, wherein each discrete fabric segment includes lateral edges extending from the first end to the second end of the discrete fabric segment,
wherein with the prosthetic heart valve device in a radially expanded deployed state, the discrete fabric segments are configured to move circumferentially relative to each other such that adjacent lateral edges of adjacent fabric segments move between a first overlapping configuration in which the adjacent lateral edges overlap each other by a first distance and a second configuration in which the adjacent lateral edges overlap each other by a second distance smaller than the first distance or do not overlap each other, in response to forces exerted on the extension member.

2. The prosthetic valve of claim 1, wherein the adjacent lateral edges of the adjacent fabric segments are not attached to each other.

3. The prosthetic valve of claim 1, wherein the adjacent fabric segments are loosely coupled together.

4. The prosthetic valve of claim 3, wherein the adjacent fabric segments are loosely coupled together via an elastic.

5. The prosthetic valve of claim 3, wherein the adjacent fabric segments are loosely coupled together via sutures.

6. The prosthetic valve of claim 1, wherein the extension member further includes a structural member.

7. The prosthetic valve of claim 6, wherein the structural member is a wire.

8. The prosthetic valve of claim 1, wherein the anchoring member further includes a sealing member coupled to the fixation frame, wherein the extension member is attached to the sealing member such that the extension member is not directly attached to the fixation frame.

9. The prosthetic valve of claim 1, wherein the extension member is directly attached to the fixation frame.

10. The prosthetic valve of claim 1, wherein the adjacent lateral edges are attached to each other via a flexible portion, and wherein in the second configuration the adjacent later edges are spaced apart from each other by no more than a length of the flexible portion.

11. The prosthetic valve of claim 1, wherein the fabric segments do not include a separate structural member.

12. The prosthetic valve of claim 1, wherein the second ends of the fabric segments coincide with an outer circumference of the extension member.
